(19) United States
(12) Patent Application Publication
(10) Pub. No.: US 2025/0025060 A1
(43) Pub. Date: Jan. 23, 2025

(54) Heart rate monitoring method, device and apparatus

(71) Applicant: Shenzhen Smart Care Technology Co., Ltd., Shenzhen (CN)

(72) Inventor: Jun HU, Shenzhen (CN)

(73) Assignee: Shenzhen Smart Care Technology Co., Ltd., Shenzhen (CN)

(21) Appl. No.: 18/769,230

(22) Filed: Jul. 10, 2024

(30) Foreign Application Priority Data
Jul. 19, 2023 (CN) 202310882827.9

(51) Int. Cl. A61B 5/0245; A61B 5/00; A61B 5/352; G16H 15/00; G16H 50/30

(52) U.S. Cl. CPC A61B 5/0245; A61B 5/352; A61B 5/7207; A61B 5/7257; A61B 5/726; A61B 5/7267; A61B 5/7278; G16H 15/00; G16H 50/30; A61B 2562/0219

(57) ABSTRACT

The present invention relates to the technical field of heart rate monitoring, discloses a heart rate monitoring method, wherein the heart rate monitoring method comprises: obtaining initial electrocardio signals of a user by a preset heart rate sensor, and acquiring initial acceleration energies of the user by a preset acceleration sensor; pre-treating the initial electrocardio signals, obtaining pretreated target electrocardio signals, and pre-treating the initial acceleration energies to obtain pretreated target acceleration energies, the present invention can be used to help doctors and users to know current heart rate conditions, make personalized and well-targeted health management measures and improve living quality.

### TECHNICAL FIELD

The present invention relates to the technical field of heart rate monitoring, especially a heart rate monitoring method, device and apparatus.

### BACKGROUND TECHNOLOGY

With the acceleration of living rhythms, cardiovascular diseases have gradually become primary problems affecting our wellbeing. Therefore, real-time and effective heart rate monitoring is of high significance to prevention of cardiovascular diseases. However, the heart rate monitoring techniques currently available are subjected to interferences of environments, devices or personal differences, consequently, accuracy and reliability of the heart rate data is affected. To address these problems, a novel heart rate monitoring method is to be studied to improve accuracy and reliability of heart rate monitoring by pre-treatment and analysis of electrocardio signals and acceleration energies and realize effective evaluation and management of cardiovascular health conditions.

### SUMMARY OF INVENTION

The present invention provides a heart rate monitoring method, device and apparatus, to address the technical problems mentioned in the foregoing paragraphs.

A first aspect of the present invention provides a heart rate monitoring method, wherein the heart rate monitoring method comprises:

Obtaining initial electrocardio signals of a user by a preset heart rate sensor, and acquiring initial acceleration energies of the user by a preset acceleration sensor; pre-treating the initial electrocardio signals, obtaining pretreated target electrocardio signals, and pre-treating the initial acceleration energies to obtain pretreated target acceleration energies;

Positioning R-waves in the target electrocardio signals based on a preset detection algorithm, calculating time intervals in between neighboring R-waves, and obtaining R-R interval sequences, and converting the R-R interval sequences to be heart rate time series; analyzing and treating the heart rate time series as per preset indicator calculation algorithms and obtaining indicator analysis results;

Labeling the indicator analysis results and the target acceleration energies, inputting samples of the indicator analysis results after labeling and samples of the target acceleration energies after labeling into a trained heart rate monitoring model for target heart rate mode generation, and obtaining a target heart rate mode; wherein, the target heart rate mode comprises a regular heart rate waveform generated for evaluating actual electrocardio signals;

Obtaining actual heart rate signals, and calculating a relevance score between the actual heart rate signals and the target heart rate mode in a time domain or frequency domain via a preset abnormality score algorithm, and obtaining single abnormality score data, normalizing each of the abnormality score data, and obtaining unified abnormality score data, conducting weighted calculation for each of the unified abnormality score data and obtaining comprehensive abnormality score data, generating a heart rate abnormality detection report according to the comprehensive abnormality score data, and generating corresponding health management measures via the heart rate abnormality detection report.

Optionally, in a first embodiment of the first aspect of the present invention, obtaining the initial electrocardio signals of the user via the preset heart rate sensor, and obtaining the initial acceleration energies of the user by the preset acceleration sensor; treating the initial electrocardio signals and obtaining the pretreated target electrocardio signals and pretreating the initial acceleration energies and obtaining the pre-treated target acceleration energies, comprising:

Acquiring the initial electrocardio signals via the preset heart rate sensor, and obtaining the initial acceleration energies of the user by the preset acceleration sensor; giving wavelet decomposition to respectively the initial electrocardio signals and the initial acceleration energies, and obtaining respective original wavelet coefficients thereof;

Building an integrated learning framework, in view of historical data and real-time operation data, self-adaptively optimizing parameters of conventional wavelet threshold functions, training using a plurality of base learners and obtaining a plurality of wavelet threshold functions with different accuracies;

Calculating optimum combinational weights of parameters of the wavelet threshold functions corresponding to each of the plurality of base learners via a preset particle swarm algorithm and generating self-adaptive wavelet threshold functions;

Substituting the obtained original wavelet coefficients of initial electrocardio signals and the initial acceleration energies respectively into the generated self-adaptive wavelet threshold functions, giving threshold treatment to the original wavelet coefficients using unified threshold methods, and obtaining the electrocardio signal and acceleration energy wavelet coefficients after threshold treatment;

Reconstructing the electrocardio signal and acceleration energy wavelet coefficients after threshold treatment and obtaining reconstructed target electrocardio signals and target acceleration energies.

Optionally, in a second embodiment of the first aspect of the present invention, positioning the R-waves in the target electrocardio signals based on the preset detection algorithm, calculating the time intervals between neighboring R-waves and obtaining the RR interval sequences and converting the RR interval sequence into the heart rate time series; analyzing and treating the heart rate time series via the preset indicator calculation algorithm and obtaining the indicator analysis results, comprising:

Positioning the R-waves in the target electrocardio signals based on the preset detection algorithm, calculating the time interval in between neighboring R-waves, and obtaining the RR interval sequences, and converting the RR interval sequences into the heart rate time series;

Conducting feature extraction for multi-parameter heart rate time series, and obtaining a plurality of feature data, wherein, the plurality of feature data comprise features in time, frequency and time-frequency domains;

Analyzing and treating the plurality of feature data according to the preset indicator calculation algorithm, and obtaining the indicator analysis results.

Optionally, in a third embodiment of the present invention, training of the heart rate monitoring model comprising:

Obtaining heterogeneous data of heart rate biological signal data related to user behaviors and environments, pretreating and encoding the heart rate biological signal data and the heterogeneous data, conducting key feature extraction for the pretreated and encoded heart rate biological signal data and heterogeneous data respectively via non-linear dimensionality reduction algorithms, obtaining heart rate feature vectors and heterogeneous feature vectors, concatenating the heart rate feature vectors and the heterogeneous feature vectors, obtaining a first training feature vector, labeling the first training feature vector, obtaining samples of the first training feature vector; wherein, the heart rate biological signal data and the heterogeneous data correlated to the user behaviors and environments serve as initial training data for the heart rate monitoring model;

Building a heart rate monitoring model, inputting the first training feature vector into a four-layer convolutional network layers for training and obtaining a first heart rate monitoring vector; wherein, the heart rate monitoring model comprises four-layer convolutional networks, six-layer pooling layers, three-layer residual layers, and an activation function layer;

In conjunction with biological parameters of the user and environment factors, concatenating the first training feature vector and the first heart rate monitoring vector by a preset first concatenation algorithm, and obtaining a second heart rate feature vector, inputting the second heart rate feature vector into the six-layer pooling layer of the heart rate monitoring model for training, and obtaining the second heart rate monitoring vector;

In conjunction with log data and behavior data of the user, concatenating the second heart rate feature vector and the second heart rate monitoring vector by the preset second concatenation algorithm, and obtaining a third heart rate feature vector, inputting the third heart rate feature vector into the three-layer residual layer of the heart rate monitoring model for training and obtaining a third heart rate monitoring vector;

Iterating model parameters in the heart rate monitoring model sequentially, until convergence of the activation function layer, completing model training and obtaining trained heart rate monitoring model; wherein, during model training, obtaining the trained heart rate monitoring model by conducting model parameter training by cascading of different convolution methods, training the heart rate monitoring model in different circumstances and training with a Grid Search algorithm to optimize model parameters.

Optionally, in a fourth embodiment of the first aspect of the present invention, obtaining the actual heart rate signals, calculating the relevance scores between the actual heart rate signals and the target heart rate model in the time domain or the frequency domain by the preset abnormality score detection algorithm and obtaining the abnormality score data comprising:

Pretreating the actual heart rate signals and the target heart rate mode and obtaining denoised actual heart rate signals and denoised target heart rate mode;

Calculating a mean vector of the denoised actual heart rate signal and the denoised target heart rate mode in the time sequence, obtaining first time series data and second time series data; wherein the first time series data and the second time series data are obtained by calculating means of each of the data points in a window of a fixed length;

Calculating corresponding covariance matrix based on the first time series data and the second time series data; wherein the covariance matrix is configured to evaluate relevance in between each of the time points;

Configuring a weight coefficient x and a weight coefficient B, selecting non-linear functions f and g, setting parameters p and q for the non-linear functions, meanwhile, setting an inverse function h acting on the covariance matrix and an inverse parameter r influencing the covariance matrix, and a function k and a parameter Z acting on calculation of an entire Mahalanobis distance;

Calculating the Mahalanobis distance according to the previously set weight coefficient, the non-linear function and the parameters utilizing the mean vectors, the covariance matrix of the actual heart rate signals and the target heart rate mode and differences therebetween;

Evaluating a relationship between the Mahalanobis distance and the preset threshold, determining abnormality scores in between the actual heart rate signals and the target heart rate mode according to a magnitude of the Mahalanobis distance, analyzing the abnormality scores and obtaining the abnormality score evaluation data.

Optionally, in a fifth embodiment of the first aspect of the present invention, calculating the Mahalanobis distance as per the following equation:

$$Mahalanobis distance = \sqrt{{( f ( α X , p ) - g ( β Y , q ) )}^{T} h ( {C}^{- 1} , r ) k ( Z ) ( f ( α X , p ) - g ( β Y , q ) )}$$

Wherein, X stands for the mean vector of the actual heart rate signals;

Y stands for the mean vector of the target heart rate signals;

C⁻¹ stands for an inverse of the covariance matrix;

α and β stand for the weight coefficient for adjusting the actual heart rate signals and the target heart rate signals;

F and g are the non-linear functions applied for the actual heart rate signals and the target heart rate signals, and the non-linear functions at least comprise sigmoid and tanh activation function;

P and q stand for parameters of the non-linear functions for adjusting the actual heart rate signals and the target heart rate signals;

H is the inverse function acting on the covariance matrix, and the inverse function acting on the covariance matrix comprises linear or non-linear functions;

R is a parameter affecting the inverse function influencing the covariance matrix;

K is a function acting on calculation processes of the entire Mahalanobis distance;

Z is a parameter acting on the k function;

(f(α(X), p)−g(β(Y), q))T stands for transposition of weighted non-linear vectorial differences.

Optionally, in a sixth embodiment of the first aspect of the present invention, wherein obtaining the actual heart rate signals, calculating the relevance score between the actual heart rate signals and the target heart rate mode in the time domain or the frequency domain by the preset abnormality score calculation algorithm and obtaining the abnormality measurement data comprising:

Treating the actual heart rate signals and the target heart rate signals via short-time Fourier Transform (STFT), and obtaining an STFT matrix corresponding to the actual heart rate signals and the target heart rate mode;

Calculating weighted abnormality scores between the actual heart rate signals and the target heart rate mode window by window by a weighted exponential smoothing Euclidean distance formula; wherein different weighted coefficients of the heart rate signals are set according to different frequency points;

Smoothing the weighted abnormality scores according to an exponential smoothing factor α, obtaining values predicted by exponential smoothing, wherein the exponential smoothing factor α ranges from 0-1;

Comparing the weighted abnormality scores of the actual heart rate signals and the target heart rate mode in the frequency domain for each of the windows with a preset threshold and a predicted value; where the score exceeds the threshold and the predicted value, judging the actual heart rate signals corresponding to the present window exists temporary abnormality;

Summating the abnormality scores in all the windows in the entire time series, and obtaining a set of abnormality measurement data.

Optionally, in a sixth embodiment of the first aspect of the present invention, the weighted exponential smoothing Euclidean formula comprises:

$$Abnormality Score = \sum [ α * ( \sum {[ ( {ω}_{k} * {\vert {H}_{i} ( k ) - {T}_{i} ( k ) \vert}^{2} ) / M ]}^{( \frac{1}{2} )} + ( 1 - α ) * Abnormality {Score}_{( i - 1 )} ]$$

Where Hᵢ(k) stands for values of the STFT matrix of the actual heart rate signals in the kth frequency point of the ith window;

Tᵢ(k) stands for values of the STFT matrix of the target heart rate mode in the kth frequency point of the ith window;

M is a summation of the frequency points;

ωₖ is a weighted coefficient of the kth frequency point, which is adjustable according to importance of the heart rate signals in different frequencies;

α is the exponential smoothing factor, falling into a range of 0 to 1;

Abnormality_score is the weighted abnormality score between the actual heart rate signals and the target heart rate mode in the frequency domain at the current moment;

Abnormality Score₍ᵢ₋₁₎ is the weighted abnormality score between the actual heart rate signals and the target heart rate mode in the frequency domain at the last moment;

A second aspect of the present invention provides a heart rate monitoring device, wherein the heart rate monitoring apparatus comprising:

An acquisition module, configured to obtain the initial electrocardio signals of the user via the preset heart rate sensor, and acquire the initial acceleration energies of the user via the preset acceleration sensor; pretreat the initial electrocardio signals and obtain the pretreated target electrocardio signals; and pretreat the initial acceleration energies and obtain the pretreated target acceleration energies;

A treatment module, configured to position the R-waves in the target electrocardio signals based on the preset detection algorithm, and calculate time intervals in between neighboring R-waves, obtain the RR interval sequences and convert the RR interval sequences to be heart rate time series; and analyze and treat the heart rate time series via the preset indicator calculation algorithm and obtain the indicator analysis results;

A training module, configured to label respectively the indicator analysis results and the target acceleration energies, and input the samples of the indicator analysis results formed after labeling and the samples of the target acceleration energies formed after labeling into the trained heart rate training model for generating the target heart rate mode, and obtaining the target heart rate mode; wherein the target heart rate mode comprises generated normal heart rate waveform, configured to evaluate the actual heart rate signals;

A health management measure generation module, configured to acquire the actual heart rate signals, and calculate the relevance scores in between the actual heart rate signals and the target heart rate mode in the time domain or the frequency domain by the preset abnormality score measurement algorithm, obtain the abnormality score data, normalize the abnormality score data and obtain the unified abnormality score measurement data, conduct weighted calculation for the unified abnormality score measurement data, obtain the comprehensive abnormality score data, generate the heart rate abnormality detection report according to the comprehensive abnormality score measurement data and generate corresponding health management measures via the heart rate abnormality detection report.

A third aspect of the present invention provides a heart monitoring apparatus, comprising a memory and at least one processor, wherein instructions are stored in the memory; the at least one processor calls the instructions in the memory to have the heart rate monitoring device execute the foregoing heart rate monitoring method.

Beneficial effects of the technical solutions of the present invention are: the present invention provides a heart rate monitoring method, apparatus and device. First of all, the initial electrocardio signals and the initial acceleration energies are collected by the preset heart rate sensor and the acceleration sensor, and the signals are pretreated. Subsequently, the R-waves are positioned according to the preset detection algorithm and the RR interval sequences are calculated so as to obtain the heart rate time sequences. The heart rate time sequences are analyzed as per the preset indicator calculation algorithm, and the indicator analysis results are obtained. The samples of the indicator analysis results and the samples of the target acceleration energies are input into the trained heart rate monitoring model to generate the target heart rate mode for evaluating the actual heart rate signals. The heart rate abnormality detection report is generated by comparing the relevance scores in between the actual heart rate signals and the target heart rate mode, and corresponding health management measures are taken. In the present invention, by advance treatment of the electrocardio signals and the acceleration energies, influences of environment interference and device tolerances on the heart rate monitoring will be reduced and accuracy and reliability of the heart rate data will be improved. And by evaluating the actual heart rate signals in view of the target heart rate mode, the heart rate abnormality can be identified effectively, and prevention and management of cardiovascular diseases can be improved. And with the generated heart rate abnormality detection report the current heart rate conditions can be known to a better extent and personalized and well-targeted health measurement measures can be taken to improve living quality.

### BRIEF DESCRIPTION OF DRAWINGS

FIG. 1 is a schematic diagram showing a heart rate monitoring method according to an embodiment of the present invention;

FIG. 2 is a schematic diagram showing a heart rate monitoring device according to an embodiment of the present invention.

### EMBODIMENTS

Embodiments of the present invention provide a heart rate monitoring method, apparatus and device. Terms “first”, “second”, “third”, “fourth” etc. (if any) in the specification, claims and the drawings are employed to differentiate similar objects, rather than to describe specific orders or sequences. It shall be understood that, the data used in such circumstances are mutually replaceable, so as to have the embodiments described herein to be implemented in sequences other than those shown or described here. Further, terms “comprising” or “including” and any variants thereof, intend to cover non-exclusive inclusion, for example, a process, method, system, product or device comprising a series of steps or units is not necessarily limited or the steps or units explicitly listed and can include other steps or units not clearly listed or those are intrinsic to the process, method, product or device.

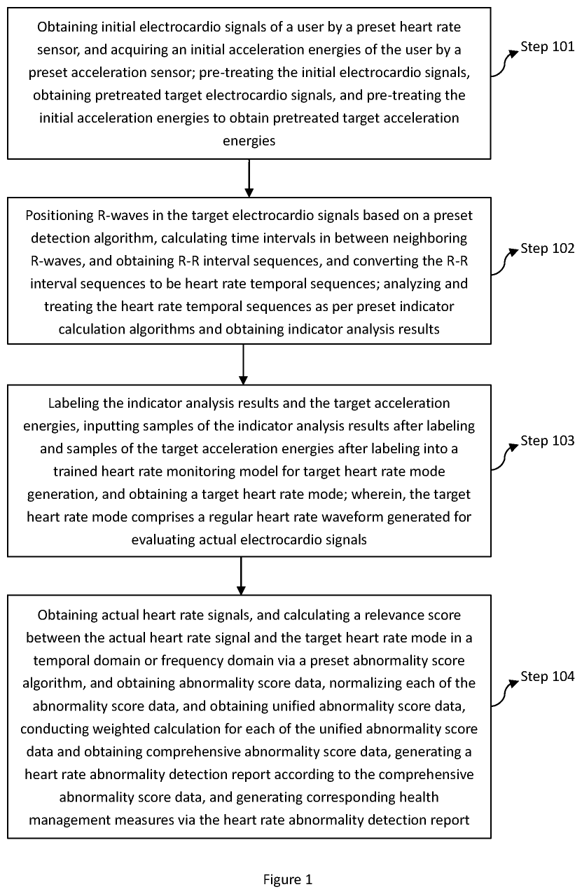

To ease understanding, hereinafter a description will be given to specific flow processes in an embodiment of the present invention, with reference to FIG. 1, an embodiment of the heart rate monitoring method comprises:

Step 101: acquiring initial electrocardio signals of a user via a preset heart rate sensor, obtaining initial acceleration energies of the user via a preset acceleration sensor; pretreating the initial electrocardio signals, obtaining pretreated target electrocardio signals, pretreating initial acceleration energies and obtaining pretreated acceleration energies;

It shall be comprehensible that, an execution party of the present invention can be a heart rate monitoring apparatus, a terminal or a server, the present invention does not limit this. The present embodiment is explained by employing a server as the execution party.

Specifically, hereinafter a detailed description will be given to the present embodiment in conjunction with specific application circumstances of an embodiment of the present invention:

A user wears an intelligent bracelet or an intelligent watch with a preset heart rate sensor and an acceleration sensor. When the user starts jogging, the heart rate sensor will capture the electrocardio signals of the user and the acceleration sensor will monitor the exercise strength of the user.

During exercise, the electrocardio signals of the user are influenced by body movement interference and noise in the surrounding environment. To ensure analysis accuracy, first of all, all the original signals are to be pretreated.

Initial electrocardio signals and acceleration energies pretreating: as the electrocardio signals and the acceleration energies are subjected to foreign interferences (for example, environment noises and sport interferences of the user etc.), big tolerances may occur if the initial signals are directly used for data analysis. Therefore, before going to subsequent analysis, the initial electrocardio signals and the acceleration energies are to be pretreated. Pretreating includes denoise, filtering, amplification, downsampling etc., aiming to eliminate interference signals and give prominence to key signal components.

Obtaining the target electrocardio signals and the target acceleration energies: after pretreating, obtaining the target electrocardio signals and the target acceleration energies. The target electrocardio signals reflect status of heart activities of the user at a specific time point. The target acceleration energies are configured to differentiate different conditions of the heart rate of the user, for example, tranquillization, exercise etc.

Step 102: positioning R-waves in the target electrocardio signals based on a preset detection algorithm, calculating time intervals in between neighboring R-waves, obtaining RR interval sequences, and converting the RR interval sequences to be heart rate time sequences; analyzing and treating the heart rate time sequences as per a preset indicator calculation algorithm and obtaining indicator analysis results;

Specifically, a further explanation on analysis and treatment using the preset detection algorithm and the indicator calculation algorithm:

Positioning the R-waves using the preset detection algorithm: first of all, identifying and positioning the R-waves based on the target electrocardio signals and the preset detection algorithm. Commonly seen R-wave detection algorithms comprise peak detection, template matching and smoothing etc.

Analyzing and treating the heart rate time sequences using the preset indicator calculation algorithm: the preset indicator calculation algorithm comprises time domain and frequency domain analysis method. The time domain analysis usually concentrates on statistical indicators of heart rate fluctuations, for example, average heart rates and standard deviations. Frequency domain analysis concentrates in frequency spectra of the heart rate fluctuations, for example, high frequency portions and low frequency portions. The indicator analysis results can be used to identify heart rate abnormality, irregular pulses or functions and status of the cardiovascular system.

Step 103: labeling respectively the indicator analysis results and the target acceleration energies, inputting the labeled samples of the indicator analysis results and the labeled samples of the target acceleration energies into a trained heart rate monitoring model for generating the target heart rate mode, and obtaining the target heart rate mode; wherein the target heart rate mode comprises generated normal heart rate waveforms, for evaluating the actual heart rate signals;

Specifically, a further description on the present embodiment is given in the following paragraphs:

Labeling the indicator analysis results and the target acceleration energies: labeling the samples is for the purpose of training the heart rate monitoring model. Labeling relates to assigning an appropriate label for each of the indicator analysis results and the target acceleration energies. The labels can be used to represent different pulses, exercise strength etc.

Training the heart rate monitoring model: training the heart rate monitoring model by machine learning algorithm (for example, support vector machines, neural network etc.) based on the labels.

Generating the target heart rate mode: inferring the target heart rate mode of the user in different conditions with the trained heart rate monitoring model according to the samples of the indicator analysis results and the samples of the target acceleration energies. The target heart rate mode comprises a normal heart rate waveform based on model prediction, which is contributive to evaluating abnormality of the actual heart rate signals.

Evaluating the actual heart rate signals: determining differences in between the actual heart rate signals and the expected results by comparing the actual heart rate signals and the generated target heart rate mode. This is contributive to judging whether abnormal waveforms or irregular pulses appear in the actual signals. If the abnormal waveforms are detected, attention shall be paid during monitoring or measures taken in order to maintain cardiovascular health.

Step 104: acquiring the actual heart rate signals, calculating the relevance scores between the actual heart rate signals and the target heart rate mode in the time series or the frequency domain via the preset abnormality score algorithm, obtaining single abnormality measurement data, normalizing the single abnormality measurement data, obtaining unified abnormality measurement data, conducting weighted calculation for the unified abnormality measurement data, obtaining comprehensive abnormality measurement data, generating the heart rate abnormality detection report according to the comprehensive abnormality measurement data, and generating corresponding health management measures based on the heart rate abnormality detection report.

Specifically, in the following paragraphs, a further description on the present embodiment is given:

Acquiring the actual heart rate signals: first of all, collecting the actual heart rate signals of the user in a time period. The signals can come from the real-time electrocardio graph data or the intelligent bracelet which contains a heart rate monitoring sensor.

Calculating relevance scores: analyzing the relevance scores in between the actual heart rate signals and the target heart rate mode as per a preset abnormality measurement algorithm. Conducting relevance comparison on the time series or the frequency domain. The time series analysis concentrates in overall waveforms of the signals, while the frequency domain analysis studies different frequency components of the heart rate waveforms. The relevance scores can reflect similarity in between the actual heart rate signals and the target heart rate mode.

Normalizing: in order to compare the single abnormality measurement data on the same scale, the data are to be normalized. In this way, all the single abnormality measurement data can be mapped to a predefined unified scope (in between 0 to 1).

Weighted calculation: giving weighted calculation on the unified abnormality measurement data according to importance of the unified abnormality measurement data. This means that, we can assign the weights of each of the abnormality measurement data, concatenate the weighted data, and obtain the comprehensive abnormality measurement data. This is contributive to integrating a plurality of measurement data to make the diagnosis results accurate and have a wide coverage.

Generating the heart rate abnormality detection report: the heart rate abnormality detection report can be generated based on the calculated comprehensive abnormality measurement data. The report describes in detail the differences between the actual heart rate signals and the target heart rate mode, and reveals potential irregular pulses, racing hearts or bradycardia.

Corresponding health management measures: according to the heart rate abnormality detection report, well-targeted health management advice can be prepared for the user. For example, where the report tells a racing heart, the advice is to reduce the exercise strength; where the report tells the irregular pulse, the advice is to seek professional medical help.

In the present embodiment, the beneficial effects are: the present invention provides a heart rate monitoring method, first of all, the initial electrocardio signals and the initial acceleration energies are acquired respectively by the preset heart rate sensor and the acceleration sensor and the signals are pretreated. Subsequently, positioning the R-waves as per the preset detection algorithm, and calculating the RR interval sequences, and obtaining the heart rate time series. Analyzing the heart rate time series, as per the preset indicator calculation algorithm and obtaining the indicator analysis results. Inputting the samples of the indicator analysis results and the samples of the target acceleration energies into the trained heart rate monitoring model for generating the target heart rate mode in order to evaluate the actual heart rate signals. By comparing the relevance scores in between the actual heart rate signals and the target heart rate signals, generating the heart rate abnormality detection report and making corresponding health management measures. In the present invention, by pretreating of the electrocardio signals and the acceleration energies, influences of environment interference and instrumental tolerances on the heart rate monitoring has been reduced, in this way, accuracy and reliability of the heart rate data is improved. And by evaluating the actual heart rate signals in view of the target heart rate mode, heart rate abnormality can be identified effectively and prevention and management abilities of cardiovascular diseases are improved. Further, the heart rate abnormality detection report can be used to help the doctors and the users to know the current heart rate conditions, make personalized and well-targeted health management measures and improve living quality. Another embodiment of the heart rate monitoring method in an embodiment of the present invention comprises:

Obtaining initial electrocardio signals of a user via a preset heart rate sensor, obtaining initial acceleration energies of the user via a preset acceleration sensor; pretreating the initial electrocardio signals, obtaining the pretreated target electrocardio signals, pretreating the initial acceleration energies, and obtaining the pretreated target acceleration energies, comprising:

Acquiring the initial electrocardio signals of the user via the preset heart rate sensor, acquiring the initial acceleration energies of the user via the preset acceleration sensor; wavelet decomposing the initial electrocardio signals and the initial acceleration energies via a preset wavelet basis function and decomposition layers, and obtaining original wavelet coefficients;

Building an integrated learning architecture, in view of historical data and real-time operation data, self-adaptively optimizing parameters of conventional wavelet threshold functions, training using a plurality of basis learners, and obtaining wavelet threshold functions with different accuracies;

Calculating optimum combinational weights of the parameters of the wavelet threshold functions corresponding to the basis learners via a preset particle clustering algorithm and generating self-adaptive wavelet threshold functions;

Substituting the original wavelet coefficients of the initial electrocardio signals and the initial acceleration energies into the generated self-adaptive wavelet threshold function, giving threshold treatment to the original wavelet coefficients using a unified threshold algorithm, and obtaining the electrocardio signal and acceleration energies wavelet coefficients after threshold treatment;

Reconstructing the electrocardio signal and acceleration energy wavelet coefficients after threshold treatment, and obtaining the reconstructed target electrocardio signal and the target acceleration energies.

Specifically, here is a further explanation and example on the present embodiment:

Acquiring the initial data: acquiring the initial electrocardio signals of the user via the preset heart rate sensor, and obtaining the initial acceleration energies of the user via the acceleration sensor. The heart rate sensor usually can be found in the intelligent bracelet or the ECG devices, and the acceleration sensor is obtainable during movement monitoring.

Conducting wavelet decomposition: wavelet decomposing the initial electrcardio signals and the initial acceleration energies by the preset wavelet basis function and the decomposition layers, and obtaining the original wavelet coefficients. For wavelet decomposition the original signals are analyzed in a plurality of scales, so as to extract local features of the signals for subsequent analysis.

For example, where the initial electrocardio signals have different frequency components, by wavelet decomposition, the different frequency components can be separated and meanful information can be extracted.

Building an integrated learning architecture: building a self-adaptive integrated learning architecture by combining the historical data and the real-time operation data. Training using a plurality of basis learners to optimize the parameters of the conventional wavelet threshold function. In this way accuracy of the wavelet threshold treatment can be improved.

Calculating optimum weights: calculating the optimum combination weights of the parameters of the wavelet threshold functions corresponding to each of the basis learners via the preset particle clustering algorithm. This is contributive to generating self-adaptive wavelet threshold functions, and dynamic adjustment can be made in view of different signal features. For example, for irregular pulse, the weights will be automatically adjusted to reserve corresponding abnormality information.

Conducting threshold treatment: substituting the original wavelet coefficients in the generated self-adaptive wavelet threshold functions, and giving threshold treatment to the original wavelet coefficients by the unified threshold algorithm. By this way the noise level can be reduced and signal quality can be improved.

Signal reconstruction: reconstructing the wavelet coefficients of the electrocardio signals and the acceleration energies after threshold treatment, and obtaining the reconstructed target electrocardio signals and the target acceleration energies. The signals can be used for subsequent heart rate monitoring model training and abnormality detection.

The beneficial effects of the present embodiment of the present invention are: in the present embodiment, by wavelet analysis, the integrated learning architecture, threshold treatment and reconstruction, the accuracy of the initial electrocardio signals and the initial acceleration energies is improved, and forceful support is provided for further heart rate diagnosis and health evaluation.

Another embodiment of the heart rate monitoring method according to the present invention comprises:

Positioning the R-waves in the target electrocardio signals based on the preset detection algorithm, calculating time intervals in between neighboring R-waves, obtaining RR interval sequences, converting the RR interval sequences to be heart rate time series; analyzing and treating the heart rate time series via the preset indicator calculation algorithm, and obtaining the indicator analysis results, comprising:

Positioning the R-waves in the target electrocardio signals based on the preset detection algorithm, calculating the time intervals in between neighboring R-waves, obtaining the RR interval sequences, and converting the RR interval sequences into the heart rate time series;

Acquiring biological data and environment parameter data with a preset biological sensor, constructing multi-parameter time series according to the biological data, the environment parameter data and the heart rate time series, wherein the biological data comprises biological signals other than the target electrocardio signals;

Conducting feature extraction for the multi-parameter heart rate time series, and obtaining a plurality of feature data, wherein the plurality of feature data comprise time, frequency and time-frequency domain features;

Analyzing and treating the plurality of feature data according to the preset indicator calculation algorithm, and obtaining the indicator analysis results.

Specifically, hereinafter a further description and example of the present embodiment will be given:

Positioning the R-waves and calculating the RR interval sequences: first of all, positioning the R-waves in the target electrocardio signals according to the preset detection algorithm. The R-waves comprise a peak in QRS waves of the ECG graphs, and are representation of heart pulse. Subsequently, calculating the time intervals in between the neighboring R-waves (RR intervals) and converting the RR interval sequence into the heart rate time series.

Building multi-parameter heart rate time series: obtaining biological data (for example, blood pressure, blood oxygen concentrations etc.) and environment parameter data (for example, temperature, humidity etc.) via the preset biological sensor, and building a multi-parameter time series in conjunction with the heart rate time series. This is contributive to providing more comprehensive information to monitor and evaluate cardiovascular health more accurately.

Feature extraction: conducting feature extraction for the multi-parameter heart rate time series, and obtaining a plurality of feature data. The features comprise time domain features (fluctuation magnitude, wave lengths etc.), frequency domain features (for example, basic frequencies, peak frequencies) and time-frequency domain features (for example, short Fourier transform, wavelet transform etc.). The features are helpful in revealing the information correlated to cardiovascular health in the electrocardio signals.

For example, the frequency domain features can be used to evaluate heart rate changes in the heart beat cycles, and are useful for evaluating heart functions in a certain extent.

Indicator calculation and analysis: analyzing and treating the plurality of features according to the preset indicator calculation algorithm and obtaining the indicator analysis results. The results can be used to evaluate cardiovascular health conditions, detection abnormality, and provide personalized health management solutions for the users.

In the present technical solution, for the detection algorithm and the indicator calculation algorithm, hereinafter a specific explanation is given:

Detection algorithm: the detection algorithm is primarily used for positioning the R-waves in the target electrocardio signals.

The detection algorithm comprises at least the following detection algorithms:

Pan-Tompkins algorithm: an algorithm that is highly reliable and widely used for electrocardio signal analysis based on features of QRS waves.

Hamilton-Tompkins algorithm: an improvement based on Pan-Tompkins, wherein the R-wave detection performance in quick heart rate conditions is enhanced.

QRS detection using Hilbert Transform: detecting the QRS complex waves from the target electrocardio signals using Hilbert Transform.

Indicator calculation algorithms: the algorithms are configured to analyze feature data to obtain the indicator related to cardiovascular health. Specifically, the algorithms comprise:

Time domain analysis: focusing on analyzing fluctuation magnitudes, waveform length etc. of the heart rate time series, for example, average heart rates, standard deviations etc.

Frequency domain analysis: measuring heart rate changes by for example power spectral density (PSD) calculation, comprising indicators such as basic frequencies, peak frequencies etc.

Random process analysis: analyzing randomness and periodicity of the heart rate time series using algorithms such as autocorrelation function (ACF), partial autocorrelation function (PACF).

Non-linear analysis: studying on complexity and long-term dependencies using methods such as sample entropy analysis and clustered coefficient calculation.

During actual application and practice, as required or as per the features of the target signals the foregoing algorithms can be chosen or combined for R-wave detection and indicator calculation.

In the present embodiment, the beneficial effects are: in the present embodiment, by positioning the R-waves, calculating the RR interval sequences, building the multi-parameter heart rate time series in view of biological data and environment parameters, conducting feature extraction and analysis, finally the indicator analysis results are obtained, with the results, the cardiovascular health conditions can be accurately evaluated and effective health management measures can be provided.

Another embodiment of the heart rate monitoring method according to the present invention comprises:

A training process of the heart rate monitoring model, comprising:

Acquiring heterogeneous data of the heart rate biological signal data correlated to user behaviors and environments, pretreating and encoding the heart rate biological signal data and the heterogeneous data, extracting key features from the pretreated and encoded heart rate biological signal data and the heterogeneous data via non-linear dimensionality reduction algorithm, obtaining heart rate feature vectors and heterogeneous feature vectors, concatenating the heart rate feature vectors and the heterogeneous feature vectors, obtaining first training feature vectors, labeling the first training feature vectors, obtaining first training feature vector samples; wherein the heart rate biological signal data and the heterogeneous data correlated to the user behaviors and the environments are used as the initial training data for the heart rate monitoring model;

Building the heart rate monitoring model, inputting the first training feature vector samples into four layers of convolutional network layers of the heart rate monitoring model for training, obtaining first heart rate monitoring vectors; wherein the heart rate monitoring model comprises four layers of convolutional networks, six layers of pooling layers, three layers of residual layers and an activation function layer;

Concatenating the first training feature vectors and the first heart rate monitoring vectors via a preset first concatenating algorithm in conjunction with biological parameters of the user and environment factors, obtaining second heart rate feature vectors, inputting the second heart rate feature vectors into the six layers of pooling layers of the heart rate monitoring model for training, obtaining second heart rate monitoring vectors;

Concatenating the second heart rate feature vectors and the second heart rate monitoring vectors via a preset second concatenating algorithm in conjunction with log data and behavior data of the user, obtaining third heart rate feature vectors, inputting the third heart rate feature vectors into the three layers of residual layers of the heart rate monitoring model for training, and obtaining third heart rate monitoring vectors;

Iteratively adjusting parameters of the heart rate monitoring model in sequence, until convergence of the activation function layer, completing model training and obtaining the trained heart rate monitoring model; wherein during model training, model parameter training is done by cascade connection of different convolution methods, and training the heart rate monitoring model in different conditions, and training is done by using Grid Search algorithm to optimize model parameters and obtaining the trained heart rate monitoring model.

Specifically, here is a further explanation and example of the present embodiment:

Data pretreating and feature extraction: collecting heart rate biological signal data and the heterogeneous data (for example user behaviors and environment data). By pretreating and encoding the heart rate biological signal data and the heterogeneous data, computer processing can be more convenient.

Extracting key features of the pretreated and encoded data using the non-linear dimension reduction algorithm (for example principal component analysis (PCA) and t-Distributed Stochastic Neighbor Embedding (t-SNE)) and generating the heart rate feature vectors and the heterogeneous feature vectors.

Concatenating the heart rate feature vectors and the heterogeneous feature vectors and obtaining the first training feature vectors. Labeling the first training feature vectors and obtaining the first training feature vector samples.

Building the heart rate monitoring model: the model comprises four layers of convolutional networks, six layers of pooling layers, three layers of residual layers and a layer of activation function layer. Inputting the first training feature vectors into the four layers of convolutional network layers for model training and obtaining the first heart rate monitoring vectors.

Increasing biological and environment factors: in view of biological parameters of the user and environment factors, concatenating the first training feature vectors and the first heart rate monitoring vectors via the first concatenating algorithm, and obtaining the second heart rate feature vectors.

Inputting the second heart feature vectors into the six layers of pooling layers for model training and obtaining the second heart rate monitoring vectors.

Adding the user log and behavior data: in conjunction with the log data and behavior data of the user, concatenating the second heart rate feature vectors and the second heart rate monitoring vectors via the second concatenating algorithm, and obtaining third heart rate feature vectors. Inputting the third heart rate feature vectors into the three layers of residual layers for model training and obtaining the third heart rate monitoring vectors.

Model optimization: during training, by cascade connection of different convolution methods and training the heart monitoring models for different conditions the model parameters are optimized. Using the Grid Search algorithm for parameter optimization until convergence of the activation function layer, completing model training and obtaining the trained heart rate monitoring model.

In the present technical solution, the first concatenation algorithm and the second concatenation algorithm are two algorithms used for concatenation of the feature vectors. Specifically:

The first concatenation algorithm: in this stage, the first concatenation algorithm is primarily configured to concatenate the first training feature vectors and the first heart rate monitoring vectors. The algorithm can be a simple vector concatenation algorithm, for example, horizontal concatenation (connecting the feature vectors along a horizontal direction) or vertical concatenation (connecting the feature vectors along a vertical direction). Further, more complex concatenation methods can be used, for example, PCA, fusing two set of data into a feature space with a higher discrimination ability.

The second concatenation algorithm: in this stage, the second concatenation algorithm is primarily configured to concatenate the second heart rate feature vectors with the second heart rate monitoring vectors. Similar to the first concatenation algorithm, simple vector concatenation methods can be used (horizontal concatenation or vertical concatenation). When dealing with more complex conditions, other concatenation methods can be used, for example, the weighted average method, assigning different weights to the features according to degrees of importance, or using feature selection algorithms (for example, the optimal subset or recursion elimination), to build an optimal feature frame in view of features from different sources.

The beneficial effects of the present embodiment: the heart rate monitoring model in the present embodiment has combined the biological signal data and other heterogeneous data (environments, behaviors and logs) to improve heart rate monitoring accuracy. By combination of a plurality of algorithms, dimension reduction techniques and deep learning, the cardiovascular conditions of the user can be detected and analyzed more effectively, so as to provide better health management advice.

Another embodiment of the heart rate monitoring method according to the present invention comprises:

Acquiring the actual heart rate signals, calculating the relevance scores between the actual heart rate signals and the target heart rate mode via the preset abnormality measurement algorithm, and obtaining single abnormality measurement data, comprising:

Conducting data pretreatment for the actual heart rate signals and the target heart rate mode, and obtaining denoised actual heart rate signals and denoised target heart rate mode;

Calculating mean vectors of the denoised actual heart rate signals and the denoised target heart rate mode in the time series, obtaining first time series data and second time series data; wherein the first time series data and the second time series data are obtainable by calculating means of each of data points in fixed-length window;

Calculating a covariance matrix corresponding to the first time series data and the second time series data respectively; wherein the covariance matrix is employed to evaluate relevance between each of the time points;

Setting weight coefficients «x and B, selecting non-linear functions f and g, setting parameters of the non-linear functions p and q, in the meanwhile, setting inverse parameters h acting on the covariance matrix and the inverse parameter r influencing the covariance matrix, and the function k and the parameter Z acting on calculation of the Mahalanobis distance;

Utilizing the mean vectors, covariance matrix and differences between the actual heart rate signals and the target heart rate mode, calculating the Mahalanobis distance as per the weight coefficients, the non-linear functions and the parameters previously set;

Evaluating relationships between the Mahalanobis distance and the set thresholds, determining the abnormality between the actual heart rate signals and the target heart rate mode according to the magnitude of the Mahalanobis distance, analyzing and treating the abnormality, and obtaining the single abnormality measurement data.

Specifically, taken as an example of a health application: based on the expected heart rate waveform model, comparing the actual heart rate signals with the expected heart rate waveform, and finding possible abnormality or risk. By the foregoing steps, the Mahalanobis distance between the actual heart rate signals and the target heart rate mode can be calculated, data analysis can be done to assist the user to know health conditions and improve exercise plans.

Another embodiment of the heart rate monitoring method comprising:

Calculating the Mahalanobis distance as per the following equation:

$$Mahalanobis distance = \sqrt{{( f ( α X , p ) - g ( β Y , q ) )}^{T} h ( {C}^{- 1} , r ) k ( Z ) ( f ( α X , p ) - g ( β Y , q ) )}$$

Wherein X stands for the mean vectors of the actual heart rate signals;

Y stands for the mean vectors of the target heart rate mode;

C⁻¹ stands for an inverse of the covariance matrix;

α and β stand for the weight coefficients for adjusting the actual heart rate signals and the target heart rate mode;

F and g are non-linear functions applied for the actual heart rate signals and the target heart rate mode, wherein the non-linear functions comprise sigmoid, tanh activation functions;

P and q stand for parameters of the non-linear functions for adjusting the actual heart rate signals and the target heart rate mode;

H stands for an inverse function acting on the covariance matrix, and the inverse function of the covariance matrix comprises linear or non-linear functions;

R stands for parameters of the inverse functions influencing the covariance matrix;

K stands for functions acting on calculation of the entire Mahalanobis distance;

Z stands for parameters acting on the k functions;

(f(α(X), p)−g(β(Y), q))T stands for transposition of weighted non-linear vectorial differences.

The beneficial effects of the present embodiment are: in the present embodiment, by calculating the Mahalanobis distance between the actual heart rate signals and the target heart rate mode, further data analysis is done to assist the user in knowing health conditions and improving exercise plans.

With the present technical solution, by calculating the abnormality degrees between the actual heart rate signals and the target heart rate mode, the health management application or other relevant art can know to a better extent differences or abnormalities between the heart rate signals, and personalized services can be provided to the users.

Another embodiment of the heart rate monitoring method according to the present invention comprises:

Acquiring the actual heart rate signals, calculating the relevance scores between the actual heart rate signals and the target heart rate mode in the time series or frequency domain by the preset abnormality measurement algorithm, and obtaining the single abnormality measurement data; comprising:

Treating the actual heart rate signals and the target heart rate mode by short-time Fourier Transform, obtaining an STFT matrix corresponding to the actual heart rate signals and the target heart rate mode;

Calculating the weighted abnormality scores between the actual heart rate signals and the target heart rate mode in the frequency domain by the window via the weighted exponential smoothing Euclidean distance equation; wherein setting the weight coefficients of the heart rate signals as per different frequency points;

Smoothing the weighted abnormality scores according to the exponential smoothing factor α, and obtaining the predicted values predicted by exponential smoothing, wherein the exponential smoothing factor α ranges from 0-1;

Obtaining the weighted abnormality scores between the actual heart rate signals and the target heart rate mode in the frequency domain, and comparing the predefined threshold with the predicted values; where the scores go beyond the thresholds and the predicted values, judging that instant abnormality exists in the actual heart rate signals corresponding to the current window; and

Summarizing the abnormality scores of all the windows in the entire time series, and obtaining the single abnormality measurement data.

Specifically, the present technical solution describes the relevance scores in between the actual heart rate signals and the target heart rate mode in the frequency domain to obtain the single abnormality measurement data. Hereinafter a deep explanation and example on the present technical solution is given:

Short time Fourier transform (STFT): converting the actual heart rate signals and the target heart rate mode from the time domain to the frequency domain using the STFT, and obtaining the STFT matrix corresponding to the actual heart rate signals and the target heart rate mode.

Calculating weighted abnormality scores: calculating the weighted abnormality scores between the actual heart rate signals and the target heart rate mode in the frequency domain window by window using the weighted exponential smoothing Euclidean distance equation, and setting the weight coefficients for the heart rate signals according to different frequency points.

Exponential smoothing and prediction: smoothing the weighted abnormality scores according to the exponential smoothing factor α (ranging in 0-1) and obtaining the predicted values predicted by exponential smoothing.

Judging abnormality scores: for each window, after obtaining the weighted abnormality scores between the actual heart rate signals and the target heart rate mode in the frequency domain, comparing the predefined threshold with the predicted values; where the scores exceed the thresholds and the predicted values, judging the transient abnormality exists the actual heart rate signals corresponding to the window.

Summarizing and obtaining the single abnormality measurement data: summating the abnormality scores in the windows in the time series, and obtaining the single abnormality measurement data.

Another embodiment of the heart rate monitoring method in the present invention comprises:

The weighted exponential smoothing Euclidean distance equation is specifically:

$$Abnormality Score = \sum [ α * ( \sum {[ ( {ω}_{k} * {\vert {H}_{i} ( k ) - {T}_{i} ( k ) \vert}^{2} ) / M ]}^{( \frac{1}{2} )} + ( 1 - α ) * Abnormality {Score}_{( i - 1 )} ] ;$$

Where Hᵢ(k) stands for values of the STFT matrix of the actual heart rate signals in the kth frequency point of the ith window;

Tᵢ(k) stands for values of the STFT matrix of the target heart rate mode in the kth frequency point of the ith window;

M is a summation of the frequency points;

ωₖ is a weighted coefficient of the kth frequency point, which is adjustable according to importance of the heart rate signals in different frequencies;

α is the exponential smoothing factor, falling into a range of 0 to 1;

Abnormality_score is the weighted abnormality score between the actual heart rate signals and the target heart rate mode in the frequency domain at the current moment;

Abnormality Score₍ᵢ₋₁₎ is the weighted abnormality score between the actual heart rate signals and the target heart rate mode in the frequency domain at the last moment;

The beneficial effects of the present invention are: in the present embodiment, the abnormality degrees of the actual heart rates are detected and judged in the frequency domain via the target heart rate mode based on personal conditions or exercise conditions and the actual heart rates by the STFT and the weighted exponential smoothing Euclidean distance. By real-time monitoring and warning the user can know to a better extent the health conditions of himself, manage timely and avoid possible adverse health problems.

In summary, by calculating the relevance scores between the actual heart rate signals and the target heart rate mode in the present technical solution, the abnormality scores of the heart rate signals can be detected more accurately, so as to provide customized services for health monitoring and management applications.

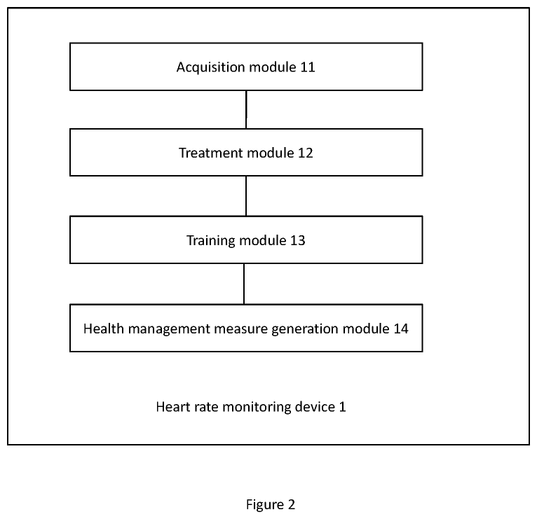

In the foregoing paragraphs, the heart rate monitoring method is described according to the embodiments of the present invention, hereinafter a description will be given to the heart rate monitoring device according to an embodiment of the present invention, with reference to FIG. 2, an embodiment of the heart rate monitoring device 1 comprising:

An acquisition module 11, configured to obtain the initial electrocardio signals of the user via the preset heart rate sensor, and acquire the initial acceleration energies of the user via the preset acceleration sensor; pretreat the initial electrocardio signals and obtain the pretreated target electrocardio signals; and pretreat the initial acceleration energies and obtain the pretreated target acceleration energies;

A treatment module 12, configured to position the R-waves in the target electrocardio signals based on the preset detection algorithm, and calculate time intervals in between neighboring R-waves, obtain the RR interval sequences and convert the RR interval sequences to be heart rate time sequences; and analyze and treat the heart rate time sequences via the preset indicator calculation algorithm and obtain the indicator analysis results;

A training module 13, configured to label respectively the indicator analysis results and the target acceleration energies, and input the samples of the indicator analysis results formed after labeling and the samples of the target acceleration energies formed after labeling into the trained heart rate training model for generating the target heart rate mode, and obtaining the target heart rate mode; wherein the target heart rate mode comprises generated normal heart rate waveform, configured to evaluate the actual heart rate signals;

A health management measure generation module 14, configured to acquire the actual heart rate signals, and calculate the relevance scores in between the actual heart rate signals and the target heart rate mode in the time domain or the frequency domain by the preset abnormality score measurement algorithm, obtain the abnormality score data, normalize the abnormality score data and obtain the unified abnormality score measurement data, conduct weighted calculation for the unified abnormality score measurement data, obtain the comprehensive abnormality score data, generate the heart rate abnormality detection report according to the comprehensive abnormality score measurement data and generate corresponding health management measures via the heart rate abnormality detection report.

In the present embodiment, for specific implementation of the modules of the heart rate monitoring device embodiment please refer to the foregoing method embodiment, which will not be repeated here.

The present invention further provides a heart rate monitoring apparatus, wherein a memory and a processor are stored in the heart rate monitoring apparatus, computer readable instructions are stored in the memory, when being executed by the processor, the computer readable instructions can have the processor execute the steps of the heart rate monitoring method as set forth in the foregoing embodiments.

Those skilled in the art shall appreciate that, to ease description, the specific working processes of the foregoing system, device and units can be the same as the corresponding processes set forth in the foregoing method embodiment, and will not be repeated again here.

Beneficial effects: the present invention provides a heart rate monitoring method, apparatus and device. First of all, the initial electrocardio signals and the initial acceleration energies are collected by the preset heart rate sensor and the acceleration sensor, and the signals are pretreated. Subsequently, the R-waves are positioned according to the preset detection algorithm and the RR interval sequences are calculated so as to obtain the heart rate time sequences. The heart rate time sequences are analyzed as per the preset indicator calculation algorithm, and the indicator analysis results are obtained. The samples of the indicator analysis results and the samples of the target acceleration energies are input into the trained heart rate monitoring model to generate the target heart rate mode for evaluating the actual heart rate signals. The heart rate abnormality detection report is generated by comparing the relevance scores in between the actual heart rate signals and the target heart rate mode, and corresponding health management measures are taken. In the present invention, by advance treatment of the electrocardio signals and the acceleration energies, influences of environment interference and device tolerances on the heart rate monitoring will be reduced and accuracy and reliability of the heart rate data will be improved. And by evaluating the actual heart rate signals in view of the target heart rate mode, heart rate abnormality can be identified effectively, and prevention and management of cardiovascular diseases can be improved. And with the generated heart rate abnormality detection report the current heart rate conditions can be known to a better extent and personalized and well-targeted health measurement measures can be taken to improve living quality.

The integrated units when being implemented in the form of software functional units and sold or used as an independent product, can be stored in a computer readable memory. Based on such understanding, the technical solutions of the present invention or all or some of the technical solutions those are contributive over the prior art can be implemented in the form of software products, the computer software product is stored in a storage medium, comprising instructions for having a computer device (a personal computer, server, or network device etc.) to execute all or some steps of the method set forth in the embodiments of the present invention. And the foregoing storage medium comprises: U-disk, hard disk, read-only memory, random access memory, magnetic disk or optical disk and a variety of medium that can be used to store program codes.

In the foregoing paragraphs the embodiments are given for explaining the technical solutions of the present invention rather than limiting the same; although a detailed description is given to the present invention with reference to the foregoing embodiments, those of ordinary skill in the art shall appreciate that, it is still possible to amend the technical solutions recited in the embodiments, or replace some technical features with equivalents; and such replacement or modification does not deviate the essence of the corresponding technical solutions from the spirit and scope of the technical solutions in the embodiments of the present invention.

1. A heart rate monitoring method, wherein the heart rate monitoring method comprises:
obtaining initial electrocardio signals of a user by a preset heart rate sensor, and acquiring initial acceleration energies of the user by a preset acceleration sensor; pre-treating the initial electrocardio signals, obtaining pretreated target electrocardio signals, and pre-treating the initial acceleration energies to obtain pretreated target acceleration energies;
positioning R-waves in the target electrocardio signals based on a preset detection algorithm, calculating time intervals in between neighboring R-waves, and obtaining R-R interval sequences, and converting the R-R interval sequences to be heart rate time series;
analyzing and treating the heart rate time series as per preset indicator calculation algorithms and obtaining indicator analysis results;
labeling the indicator analysis results and the target acceleration energies, inputting samples of the indicator analysis results after labeling and samples of the target acceleration energies after labeling into a trained heart rate monitoring model for target heart rate mode generation, and obtaining a target heart rate mode; wherein, the target heart rate mode comprises a regular heart rate waveform generated for evaluating actual electrocardio signals;
obtaining actual heart rate signals, and calculating a relevance score between the actual heart rate signals and the target heart rate mode in a time domain or frequency domain via a preset abnormality score algorithm, and obtaining single abnormality score data, normalizing each of the abnormality score data, and obtaining unified abnormality score data, conducting weighted calculation for each of the unified abnormality score data and obtaining comprehensive abnormality score data, generating a heart rate abnormality detection report according to the comprehensive abnormality score data, and generating corresponding health management measures via the heart rate abnormality detection report;
wherein obtaining the initial electrocardio signals of the user via the preset heart rate sensor, and obtaining the initial acceleration energies of the user by the preset acceleration sensor; treating the initial electrocardio signals and obtaining the pretreated target electrocardio signals and pretreating the initial acceleration energies and obtaining the pre-treated target acceleration energies, comprising:
acquiring the initial electrocardio signals via the preset heart rate sensor, and obtaining the initial acceleration energies of the user by the preset acceleration sensor; giving wavelet decomposition to respectively the initial electrocardio signals and the initial acceleration energies, and obtaining respective original wavelet coefficients thereof;
building an integrated learning framework, in view of historical data and real-time operation data, self-adaptively optimizing parameters of conventional wavelet threshold functions, training using a plurality of base learners and obtaining a plurality of wavelet threshold functions with different accuracies;
calculating optimum combinational weights of parameters of the wavelet threshold functions corresponding to each of the plurality of base learners via a preset particle swarm algorithm and generating self-adaptive wavelet threshold functions;
substituting the obtained original wavelet coefficients of initial electrocardio signals and the initial acceleration energies respectively into the generated self-adaptive wavelet threshold functions, giving threshold treatment to the original wavelet coefficients using unified threshold methods, and obtaining the electrocardio signal and acceleration energy wavelet coefficients after threshold treatment;
reconstructing the electrocardio signal and acceleration energy wavelet coefficients after threshold treatment and obtaining reconstructed target electrocardio signals and target acceleration energies;
wherein positioning the R-waves in the target electrocardio signals based on the preset detection algorithm, calculating the time intervals between neighboring R-waves and obtaining the RR interval sequences and converting the RR interval sequence into the heart rate time series; analyzing and treating the heart rate time series via the preset indicator calculation algorithm and obtaining the indicator analysis results, comprising:
positioning the R-waves in the target electrocardio signals based on the preset detection algorithm, calculating the time interval in between neighboring R-waves, and obtaining the RR interval sequences, and converting the RR interval sequences into the heart rate time series;
conducting feature extraction for multi-parameter heart rate time series, and obtaining a plurality of feature data, wherein, the plurality of feature data comprise features in time, frequency and time-frequency domains;
analyzing and treating the plurality of feature data according to the preset indicator calculation algorithm, and obtaining the indicator analysis results;
wherein training of the heart rate monitoring model comprising:
obtaining heterogeneous data of heart rate biological signal data related to user behaviors and environments, pretreating and encoding the heart rate biological signal data and the heterogeneous data, conducting key feature extraction for the pretreated and encoded heart rate biological signal data and heterogeneous data respectively via non-linear dimensionality reduction algorithms, obtaining heart rate feature vectors and heterogeneous feature vectors, concatenating the heart rate feature vectors and the heterogeneous feature vectors, obtaining a first training feature vector, labeling the first training feature vector, obtaining samples of the first training feature vector; wherein, the heart rate biological signal data and the heterogeneous data correlated to the user behaviors and environments serve as initial training data for the heart rate monitoring model;
building a heart rate monitoring model, inputting the first training feature vector into a four-layer convolutional network layers for training and obtaining a first heart rate monitoring vector; wherein, the heart rate monitoring model comprises four-layer convolutional networks, six-layer pooling layers, three-layer residual layers, and an activation function layer;
in conjunction with biological parameters of the user and environment factors, concatenating the first training feature vector and the first heart rate monitoring vector by a preset first concatenation algorithm, and obtaining a second heart rate feature vector, inputting the second heart rate feature vector into the six-layer pooling layer of the heart rate monitoring model for training, and obtaining the second heart rate monitoring vector;
in conjunction with log data and behavior data of the user, concatenating the second heart rate feature vector and the second heart rate monitoring vector by the preset second concatenation algorithm, and obtaining a third heart rate feature vector, inputting the third heart rate feature vector into the three-layer residual layer of the heart rate monitoring model for training and obtaining a third heart rate monitoring vector;
iterating model parameters in the heart rate monitoring model sequentially, until convergence of the activation function layer, completing model training and obtaining trained heart rate monitoring model; wherein, during model training, obtaining the trained heart rate monitoring model by conducting model parameter training by cascading of different convolution methods, training the heart rate monitoring model in different circumstances and training with a Grid Search algorithm to optimize model parameters;
wherein obtaining the actual heart rate signals, calculating the relevance scores between the actual heart rate signals and the target heart rate model in the time domain or the frequency domain by the preset abnormality score detection algorithm and obtaining the abnormality score data comprising:
pretreating the actual heart rate signals and the target heart rate mode and obtaining denoised actual heart rate signals and denoised target heart rate mode;
calculating a mean vector of the denoised actual heart rate signal and the denoised target heart rate mode in the time sequence, obtaining first time series data and second time series data; wherein the first time series data and the second time series data are obtained by calculating means of each of the data points in a window of a fixed length;
calculating corresponding covariance matrix based on the first time series data and the second time series data; wherein the covariance matrix is configured to evaluate relevance in between each of the time points;
configuring a weight coefficient a and a weight coefficient, selecting non-linear functions f and g, setting parameters p and q for the non-linear functions, meanwhile, setting an inverse function h acting on the covariance matrix and an inverse parameter r influencing the covariance matrix, and a function k and a parameter Z acting on calculation of an entire Mahalanobis distance;
calculating the Mahalanobis distance according to the previously set weight coefficient, the non-linear function and the parameters utilizing the mean vectors, the covariance matrix of the actual heart rate signals and the target heart rate mode and differences therebetween;
evaluating a relationship between the Mahalanobis distance and the preset threshold, determining abnormality scores in between the actual heart rate signals and the target heart rate mode according to a magnitude of the Mahalanobis distance, analyzing the abnormality scores and obtaining the abnormality score evaluation data; or
wherein obtaining the actual heart rate signals, calculating the relevance score between the actual heart rate signals and the target heart rate mode in the time domain or the frequency domain by the preset abnormality score calculation algorithm and obtaining the abnormality measurement data comprising:
treating the actual heart rate signals and the target heart rate signals via short-time Fourier Transform (STFT), and obtaining an STFT matrix corresponding to the actual heart rate signals and the target heart rate mode;
calculating weighted abnormality scores between the actual heart rate signals and the target heart rate mode window by window by a weighted exponential smoothing Euclidean distance formula; wherein different weighted coefficients of the heart rate signals are set according to different frequency points;
smoothing the weighted abnormality scores according to an exponential smoothing factor α, obtaining values predicted by exponential smoothing, wherein the exponential smoothing factor α ranges from 0-1;
comparing the weighted abnormality scores of the actual heart rate signals and the target heart rate mode in the frequency domain for each of the windows with a preset threshold and a predicted value; where the score exceeds the threshold and the predicted value, judging the actual heart rate signals corresponding to the present window exists temporary abnormality:
summating the abnormality scores in all the windows in the entire time series, and obtaining a set of abnormality measurement data.

2-5. (canceled)

6. The heart rate monitoring method according to claim 1, wherein calculating the Mahalanobis distance as per the following equation:
$$Mahalanobis distance = \sqrt{{( f ( α X , p ) - g ( β Y , q ) )}^{T} h ( {C}^{- 1} , r ) k ( Z ) ( f ( α X , p ) - g ( β Y , q ) )}$$
Wherein, X stands for the mean vector of the actual heart rate signals;
Y stands for the mean vector of the target heart rate signals;
C⁻¹ stands for an inverse of the covariance matrix;
α and β stand for the weight coefficient for adjusting the actual heart rate signals and the target heart rate signals;
f and g are the non-linear functions applied for the actual heart rate signals and the target heart rate signals, and the non-linear functions at least comprise sigmoid and tanh activation function;
p and q stand for parameters of the non-linear functions for adjusting the actual heart rate signals and the target heart rate signals;
h is the inverse function acting on the covariance matrix, and the inverse function acting on the covariance matrix comprises linear or non-linear functions;
r is a parameter affecting the inverse function influencing the covariance matrix;
k is a function acting on calculation processes of the entire Mahalanobis distance;
Z is a parameter acting on the k function;
(f(α(X), p)−g(β(Y), q))T stands for transposition of weighted non-linear vectorial differences.

7. (canceled)

8. The heart rate monitoring method according to claim 1, wherein the weighted exponential smoothing Euclidean formula comprises:
$$Abnormality Score = \sum [ α * ( \sum {[ ( {ω}_{k} * {\vert {H}_{i} ( k ) - {T}_{i} ( k ) \vert}^{2} ) / M ]}^{( \frac{1}{2} )} + ( 1 - α ) * Abnormality {Score}_{( i - 1 )} ]$$
Where Hᵢ(k) stands for values of the STFT matrix of the actual heart rate signals in the kth frequency point of the ith window;
Tᵢ(k) stands for values of the STFT matrix of the target heart rate mode in the kth frequency point of the ith window;
M is a summation of the frequency points;
ωₖ is a weighted coefficient of the kth frequency point, which is adjustable according to importance of the heart rate signals in different frequencies;
α is the exponential smoothing factor, falling into a range of 0 to 1;
Abnormality_score is the weighted abnormality score between the actual heart rate signals and the target heart rate mode in the frequency domain at the current moment;
Abnormality Score₍ᵢ₋₁₎ is the weighted abnormality score between the actual heart rate signals and the target heart rate mode in the frequency domain at the last moment.

9. A heart rate monitoring device, wherein the heart rate monitoring apparatus comprising:
an acquisition module, configured to obtain the initial electrocardio signals of the user via the preset heart rate sensor, and acquire the initial acceleration energies of the user via the preset acceleration sensor; pretreat the initial electrocardio signals and obtain the pretreated target electrocardio signals; and pretreat the initial acceleration energies and obtain the pretreated target acceleration energies;
a treatment module, configured to position the R-waves in the target electrocardio signals based on the preset detection algorithm, and calculate time intervals in between neighboring R-waves, obtain the RR interval sequences and convert the RR interval sequences to be heart rate time series; and analyze and treat the heart rate time series via the preset indicator calculation algorithm and obtain the indicator analysis results;
a training module, configured to label respectively the indicator analysis results and the target acceleration energies, and input the samples of the indicator analysis results formed after labeling and the samples of the target acceleration energies formed after labeling into the trained heart rate training model for generating the target heart rate mode, and obtaining the target heart rate mode; wherein the target heart rate mode comprises generated normal heart rate waveform, configured to evaluate the actual heart rate signals;
a health management measure generation module, configured to acquire the actual heart rate signals, and calculate the relevance scores in between the actual heart rate signals and the target heart rate mode in the time domain or the frequency domain by the preset abnormality score measurement algorithm, obtain the abnormality score data, normalize the abnormality score data and obtain the unified abnormality score measurement data, conduct weighted calculation for the unified abnormality score measurement data, obtain the comprehensive abnormality score data, generate the heart rate abnormality detection report according to the comprehensive abnormality score measurement data and generate corresponding health management measures via the heart rate abnormality detection report;
wherein the acquisition module is configured for
obtaining the initial electrocardio signals of the user via the preset heart rate sensor, and obtaining the initial acceleration energies of the user by the preset acceleration sensor; treating the initial electrocardio signals and obtaining the pretreated target electrocardio signals and pretreating the initial acceleration energies and obtaining the pre-treated target acceleration energies, comprising:
acquiring the initial electrocardio signals via the preset heart rate sensor, and obtaining the initial acceleration energies of the user by the preset acceleration sensor; giving wavelet decomposition to respectively the initial electrocardio signals and the initial acceleration energies, and obtaining respective original wavelet coefficients thereof;
building an integrated learning framework, in view of historical data and real-time operation data, self-adaptively optimizing parameters of conventional wavelet threshold functions, training using a plurality of base learners and obtaining a plurality of wavelet threshold functions with different accuracies;
calculating optimum combinational weights of parameters of the wavelet threshold functions corresponding to each of the plurality of base learners via a preset particle swarm algorithm and generating self-adaptive wavelet threshold functions;
substituting the obtained original wavelet coefficients of initial electrocardio signals and the initial acceleration energies respectively into the generated self-adaptive wavelet threshold functions, giving threshold treatment to the original wavelet coefficients using unified threshold methods, and obtaining the electrocardio signal and acceleration energy wavelet coefficients after threshold treatment;
reconstructing the electrocardio signal and acceleration energy wavelet coefficients after threshold treatment and obtaining reconstructed target electrocardio signals and target acceleration energies;
wherein the treatment module is configured for
positioning the R-waves in the target electrocardio signals based on the preset detection algorithm, calculating the time intervals between neighboring R-waves and obtaining the RR interval sequences and converting the RR interval sequence into the heart rate time series; analyzing and treating the heart rate time series via the preset indicator calculation algorithm and obtaining the indicator analysis results, comprising:
positioning the R-waves in the target electrocardio signals based on the preset detection algorithm, calculating the time interval in between neighboring R-waves, and obtaining the RR interval sequences, and converting the RR interval sequences into the heart rate time series;
conducting feature extraction for multi-parameter heart rate time series, and obtaining a plurality of feature data, wherein, the plurality of feature data comprise features in time, frequency and time-frequency domains;
analyzing and treating the plurality of feature data according to the preset indicator calculation algorithm, and obtaining the indicator analysis results;
wherein the training module is configured for
training of the heart rate monitoring model comprising:
obtaining heterogeneous data of heart rate biological signal data related to user behaviors and environments, pretreating and encoding the heart rate biological signal data and the heterogeneous data, conducting key feature extraction for the pretreated and encoded heart rate biological signal data and heterogeneous data respectively via non-linear dimensionality reduction algorithms, obtaining heart rate feature vectors and heterogeneous feature vectors, concatenating the heart rate feature vectors and the heterogeneous feature vectors, obtaining a first training feature vector, labeling the first training feature vector, obtaining samples of the first training feature vector; wherein, the heart rate biological signal data and the heterogeneous data correlated to the user behaviors and environments serve as initial training data for the heart rate monitoring model;
building a heart rate monitoring model, inputting the first training feature vector into a four-layer convolutional network layers for training and obtaining a first heart rate monitoring vector; wherein, the heart rate monitoring model comprises four-layer convolutional networks, six-layer pooling layers, three-layer residual layers, and an activation function layer;
in conjunction with biological parameters of the user and environment factors, concatenating the first training feature vector and the first heart rate monitoring vector by a preset first concatenation algorithm, and obtaining a second heart rate feature vector, inputting the second heart rate feature vector into the six-layer pooling layer of the heart rate monitoring model for training, and obtaining the second heart rate monitoring vector;
in conjunction with log data and behavior data of the user, concatenating the second heart rate feature vector and the second heart rate monitoring vector by the preset second concatenation algorithm, and obtaining a third heart rate feature vector, inputting the third heart rate feature vector into the three-layer residual layer of the heart rate monitoring model for training and obtaining a third heart rate monitoring vector;
iterating model parameters in the heart rate monitoring model sequentially, until convergence of the activation function layer, completing model training and obtaining trained heart rate monitoring model; wherein, during model training, obtaining the trained heart rate monitoring model by conducting model parameter training by cascading of different convolution methods, training the heart rate monitoring model in different circumstances and training with a Grid Search algorithm to optimize model parameters;
wherein the health management measure generation module is configured for:
pretreating the actual heart rate signals and the target heart rate mode and obtaining denoised actual heart rate signals and denoised target heart rate mode;
calculating a mean vector of the denoised actual heart rate signal and the denoised target heart rate mode in the time sequence, obtaining first time series data and second time series data; wherein the first time series data and the second time series data are obtained by calculating means of each of the data points in a window of a fixed length;
calculating corresponding covariance matrix based on the first time series data and the second time series data; wherein the covariance matrix is configured to evaluate relevance in between each of the time points;
configuring a weight coefficient α and a weight coefficient β, selecting non-linear functions f and g, setting parameters p and q for the non-linear functions, meanwhile, setting an inverse function h acting on the covariance matrix and an inverse parameter r influencing the covariance matrix, and a function k and a parameter Z acting on calculation of an entire Mahalanobis distance;
calculating the Mahalanobis distance according to the previously set weight coefficient, the non-linear function and the parameters utilizing the mean vectors, the covariance matrix of the actual heart rate signals and the target heart rate mode and differences therebetween;
evaluating a relationship between the Mahalanobis distance and the preset threshold, determining abnormality scores in between the actual heart rate signals and the target heart rate mode according to a magnitude of the Mahalanobis distance, analyzing the abnormality scores and obtaining the abnormality score evaluation data; or
wherein the health management measure generation module is configured for obtaining the actual heart rate signals, calculating the relevance score between the actual heart rate signals and the target heart rate mode in the time domain or the frequency domain by the preset abnormality score calculation algorithm and obtaining the abnormality measurement data comprising:
treating the actual heart rate signals and the target heart rate signals via short-time Fourier Transform (STFT), and obtaining an STFT matrix corresponding to the actual heart rate signals and the target heart rate mode;
calculating weighted abnormality scores between the actual heart rate signals and the target heart rate mode window by window by a weighted exponential smoothing Euclidean distance formula; wherein different weighted coefficients of the heart rate signals are set according to different frequency points;
smoothing the weighted abnormality scores according to an exponential smoothing factor α, obtaining values predicted by exponential smoothing, wherein the exponential smoothing factor α ranges from 0-1;
comparing the weighted abnormality scores of the actual heart rate signals and the target heart rate mode in the frequency domain for each of the windows with a preset threshold and a predicted value; where the score exceeds the threshold and the predicted value, judging the actual heart rate signals corresponding to the present window exists temporary abnormality;
summating the abnormality scores in all the windows in the entire time series, and obtaining a set of abnormality measurement data.

10. A heart monitoring apparatus, comprising a memory and at least one processor, wherein instructions are stored in the memory; the at least one processor calls the instructions in the memory to have the heart rate monitoring device execute the foregoing heart rate monitoring method of claim 1.

11. A heart monitoring apparatus, comprising a memory and at least one processor, wherein instructions are stored in the memory; the at least one processor calls the instructions in the memory to have the heart rate monitoring device execute the foregoing heart rate monitoring method of claim 6.

12. A heart monitoring apparatus, comprising a memory and at least one processor, wherein instructions are stored in the memory; the at least one processor calls the instructions in the memory to have the heart rate monitoring device execute the foregoing heart rate monitoring method of claim 8.
